(19) United States
(12) Patent Application Publication
(10) Pub. No.: US 2006/0039972 A1
(43) Pub. Date: Feb. 23, 2006

(54) Effervescent composition including a grape-derived component

(76) Inventors: Mary Aldritt, Excelsior, MN (US); Robert E. Lee, Hudson, WI (US); Fred Wehling, New Hope, MN (US)

Correspondence Address: ALLISON JOHNSON, P.A., LAKE CALHOUN EXECUTIVE CENTER, 3033 EXCELSIOR BLVD., SUITE 467, MINNEAPOLIS, MN, 55416, US

(21) Appl. No.: 10/922,075

(22) Filed: Aug. 19, 2004

(51) Int. Cl. A61K 36/87; A61K 9/46

(52) U.S. Cl. 424/466; 424/766

(57) ABSTRACT

The invention features an effervescent composition that includes at least one grape-derived component, at least one effervescent agent, and optionally at least one active agent, where the sum of the weight of the grape-derived component and the weight of the active agent constitute at least about 10% by weight of the composition, with the weight of the grape-derived component being greater than weight of the active agent.

### BACKGROUND

The present invention is directed to effervescent compositions that include at least one grape-derived component.

Studies show that atherosclerosis and coronary heart disease are linked to excessive consumption of dietary saturated fat and cholesterol. As consumers today trend toward diets with increased saturated fat, there is a desire to counteract the known health detriments associated with such diets.

The consumption of wine has been reported as an effective means of counteracting complications by decreasing LDL oxidation, increasing HDL cholesterol content, and decreasing platelet aggregation. It is desirable, however, to develop alternatives to wine particularly, non-alcoholic alternatives.

Formulating effervescent compositions suitable for use in beverages is extremely difficult. The various properties desired for an effervescent composition are many. Effervescent compositions preferably dissolve in water in a relatively short period of time to a composition that is aesthetically pleasing and palatable to an individual. In addition, the composition should exhibit good stability over a useful lifespan. Achieving these properties is not a simple process.

### SUMMARY OF THE INVENTION

In one aspect, the invention features an effervescent composition (e.g., a powder or a tablet) that includes at least one grape-derived component that includes greater than 100 milligrams (mg) polyphenols, at least one effervescent agent, binder, lubricant, and optionally at least one active agent, wherein at least 10% by weight of the tablet is the sum of the weight of the grape-derived component and the weight of the optional active agent.

In one embodiment, the grape-derived component includes at least about 200 milligrams polyphenols. In another embodiment, the grape-derived component includes at least about 300 milligrams polyphenols. In one embodiment, the grape-derived component includes at least about 25% dry weight polyphenols. In other embodiments, the grape-derived component includes at least about 75% dry weight polyphenols. In some embodiments, the grape-derived component includes at least about 1.5% dry weight anthocyanins.

In some embodiments, the grape-derived component is selected from grape seed extract, grape skin extract, grape juice extract, red wine extract, and combinations thereof. In other embodiments, the grape-derived component is derived from fermented grapes.

In other embodiments, the grape-derived component constitutes at least about 70% of the sum of the weight of the grape-derived component and the weight of the active agent. In another embodiment, the grape-derived component constitutes at least about 85% of the sum of the weight of the grape-derived component and the weight of the active agent.

In one embodiment, the tablet includes an active agent selected from vitamins, amino acids, pharmaceutical agents, minerals, dietary supplements, and combinations thereof. In some embodiments, the tablet includes an oil-soluble active agent. In other embodiments, the active agent includes vitamin E. In another embodiment, the tablet includes an active agent selected from vitamin C and vitamin E.

In another embodiment, the tablet exhibits a hardness of at least about 6 kilopounds and disintegrates in water having a temperature of about 25° C. in less than about 4 minutes.

In other embodiments the grape-derived component includes red wine extract, and the effervescent composition disintegrates in water to an opaque solution.

In another embodiment, the invention features an effervescent composition that includes at least one grape-derived component, at least one effervescent agent, and optionally at least one active agent, at least about 10% by weight of the composition is the sum of the weight of the grape-derived component and the weight of the active agent, and the weight of the grape-derived component being greater than weight of the active agent.

In another aspect, the invention features an airtight sealed package that includes a sachet, and an effervescent powder disposed in the sachet, the effervescent powder including at least two grape-derived components, at least one effervescent agent, and optionally at least one active agent.

In some embodiments, at least about 10% by weight of the effervescent powder is the grape-derived component and the active agent.

In other aspects the invention features a method of using an effervescent composition disclosed herein, where the method includes combining the effervescent composition and an aqueous liquid to form a beverage.

The effervescent composition imparts the health benefits associated with grape-derived components to those who consume aqueous beverages made therefrom. The effervescent composition also provides a convenient dosage form that provides an accurate predetermined amount of the grape-derived component, and is easy to administer. The effervescent composition also provides a grape-derived component in the form of a tablet or a powder that can be provided in a convenient package.

Other features and advantages of the invention will be apparent from the following description of preferred embodiments thereof and from the claims.

### GLOSSARY

In reference to the invention, these terms have the meanings set forth below:

The term “grape-derived component” means a substance obtained from at least one part of a grape including the skin, seeds, pulp, stem, and juice of a grape. The grape-derived component may be derived from fermented grapes or non-fermented grapes.

The term “polyphenol” refers a compound that includes more than one phenolic group.

The term “active agent” refers to a component, other than a grape-derived component, that provides a health benefit to an individual.

### DETAILED DESCRIPTION OF THE INVENTION

The effervescent composition includes at least one grape-derived component, an effervescent agent, and optionally at least one active agent. The sum of the weight of the grape-derived component and the weight of the optional active agent constitute at least about 10% by weight of the composition.

Grape-Derived Component

Any suitable grape-derived component can be included in the effervescent composition. Preferably, the grape-derived component is an extract including, e.g., grape seed extract, grape skin extract, grape juice extract, red wine extract, and combinations thereof. Preferably, the grape-derived component includes polyphenols. Incorporating polyphenols into effervescent compositions enables production of effervescent formulations that have beneficial health aspects typically associated with wine polyphenols. Those benefits includes, for example, decreased LDL oxidation, increased HDL cholesterol content, and decreased platelet aggregation. These effects make such compositions useful in, for example, the control of atherosclerosis and coronary heart diseases. The grape-derived component preferably includes at least about 25% dry weight polyphenols, or even at least about 75% dry weight polyphenols. Preferably the grape-derived component includes anthocyanins, which are a class of polyphenols. Useful grape-derived components include at least about 1.5% by dry weight anthocyanins.

Polyphenols act as antioxidants. They protect cells and body chemicals against damage caused by free radicals, which are reactive atoms that contribute to tissue damage in the body. When low-density lipoprotein (LDL) cholesterol is oxidized, for example, it can adhere to arteries and cause coronary heart disease. In addition to coronary heart disease, many other diseases are linked to free radical oxidation mechanisms. Those include, e.g., cancer, cataracts, and diabetes. Given the widespread mortality associated with coronary heart disease in the western world, its correlation with LDL oxidation is of particular interest.

One method for the determination of LDL oxidation in vitro is to employ the transition metal copper (specifically Cu²⁺ ions) as a catalyst to promote the oxidation of endogenous lipid hydroperoxides. Antioxidants present in LDL, especially alpha tocopherol, delay the oxidation process and produce a so-called “lag phase.” The process can be easily followed in a UV-spectrophotometer because the oxidation reaction produces conjugated dienes that can be continuously monitored at known wavelengths. Extension of LDL oxidation determinations from such analyses to in vivo prognosis, however, is not always synonymous.

Polyphenols occur abundantly in red wine and consist of a large number of different chemical substances of varying molecular weights. The classes of polyphenols present in red wine include, e.g., flavonoids (a term often used to denote polyphenols in general, but more commonly in Europe to denote only the flavones), the flavanols, proanthocyanidins (also called procyanidols, procyanins, procyanidins, and tannins), and anthocyanins. Also included within the general term polyphenols are dihydroxy- or tri-hydroxy benzoic acids and phytoalexins (e.g., resveratrol).

Not all grape-derived components contain the same amount of polyphenols or breakdown of polyphenols within. As compared to certain conventional grape fruit extracts, artificial flavorings, and sweeteners that merely contain a grape flavor, the grape-derived component preferably includes polyphenols. Thus, the grape-derived component is selected according to the desired effect.

Preferably, the grape-derived component includes at least about 25% dry weight, at least about 50% dry weight, or even at least about 75% dry weight polyphenols based. It is most preferred that a majority of the polyphenols be those most associated with reduction of LDL oxidation (e.g., flavonols and anthocyanins). Furthermore, it has been found that the overall polyphenol content of a grape-derived component can be significantly lower if the amount of specific polyphenols (e.g., anthocyanins) is sufficient. Preferably the grape-derived component includes at least about 1.5% dry weight, at least about 6% dry weight, at least about 7% dry weight, or even at least about 8% dry weight anthocyanins.

It is desirable to formulate effervescent compositions for administration of sufficient grape-derived component for improved health on a daily basis. Preferably, the amount of grape-derived component administered on a daily basis is that such from at least about 40 milligrams (mg) to about 350 mg, from about 100 mg to about 350 mg, or even from about 250 mg to about 350 mg polyphenols is administered on a daily basis through the consumption of one or more dosage forms, e.g., effervescent tablets.

The effervescent tablet includes at least one grape-derived component that includes at least about 100 mg, at least about 200 mg, or even at least about 300 mg polyphenols. Once formulated into such a convenient dosage form, the appropriate number of tablets can be administered to an individual on a daily basis as desired.

Examples of suitable grape-derived components include those grape-derived components based on a fermented grape product, such as red wine extract or concentrate. One useful red wine extract is commercially available under the trade designation MEGANATURAL Red Wine Concentrate, Code GW3050 from Polyphenolics, a division of Canandaigua Wine Company, Inc. (Madera, Calif.). The stated polyphenol content of this product is about 25% by weight and the total anthocyanins content is about 8% by weight. Another useful red wine extract is commercially available under the product designation Red Wine standardized powder extract from Brucia Plant Extracts, a division of Naturex, Inc. (Shingle Springs, Calif.). The stated polyphenol content of this product is about 30% by weight. Another useful red wine extract is commercially available under the trade designation PROVINOLS Red Wine Extract from Seppic (Paris, France).

In addition to those grape-derived component based on fermented grape products, grape-derived components based on non-fermented grape products (e.g., seed, juice, or skin) can provide the requisite polyphenols depending on their origin and subsequent processing. One useful grape seed extract is commercially available from Polyphenolics (Madera, Calif.) under the trade designation VINOX. Other suitable grape-derived components are available under the trade designations MEGANATURAL Gold Grape Seed Extract (with a stated polyphenol content of about 90% by weight) and MEGANATURAL GSKE grape skin extract (with a stated polyphenol content of about 80% by weight and a minimum anthocyanins content of about 1.5% by weight), both of which are commercially available from Polyphenolics.

Effervescent Agent

The effervescent agent is an effervescent couple that includes at least one acid and at least one base. Such effervescent couples are activated when contacted with water, e.g., when the tablet is placed in a glass of water. The water liberates the acid and base, enabling the acid and base to react with each other to produce carbon dioxide gas. Carbon dioxide gas imparts carbonation to the aqueous composition, which in addition to promoting dissolution of the effervescent composition also enhances the resulting taste of the dissolved composition.

Examples of useful acids include adipic acid, ascorbic acid (i.e., vitamin C), citric acid, fumaric acid, hexamic acid, lactic acid, malic acid, succinic acid, tartaric acid (e.g., wine acid), acid salts and acid anhydrides thereof, and mixtures thereof. Examples of useful acid anhydrides include citraconic anhydride, glucono-D-lactone, and succinic anhydride. Examples of useful acid salts include those generally classified as acid citrates, phosphates, sulfates, sulfites, and tartrates. Examples of the foregoing acid salts include, but are not limited to, potassium bitartrate, ascorbic acid monosodium citrate, sodium dihydrogen phosphate, disodium dihydrogen phosphate, sodium acid pyrophosophate, sodium acid sulfite, and combinations thereof.

Acid is preferably present in the overall effervescent composition in an amount of from about 10% by weight to about 60% by weight, from about 15% by weight to about 50% by weight, or even from about 25% by weight to about 40% by weight.

The base is preferably capable of generating carbon dioxide. Thus, carbonate bases are preferred. Examples of suitable carbonate bases include sodium bicarbonate, sodium carbonate, sodium sesquicarbonate, potassium carbonate, potassium bicarbonate, calcium carbonate, calcium bicarbonate, magnesium carbonate, magnesium oxide, sodium glycine carbonate, L-lysine carbonate, arginine carbonate, zinc carbonate, zinc oxide and mixtures thereof.

The base is preferably present in the overall effervescent composition in an amount of from about 10% by weight to about 60% by weight, from about 15% by weight to about 50% by weight, or even from about 25% by weight to about 40% by weight.

At least one component of the effervescent couple can also be an active agent. Active agents, many of which are considered acids or bases in and of themselves, are discussed further below. If the effervescent couple includes an active agent as either the acid or base component, a separate active agent need not be present in the overall effervescent composition. In certain embodiments of the invention, however, separate active agents are included. This allows for broader formulation latitude and a wider array of obtainable health benefits.

Active Agent

The “active agent” is a component known to provide a health benefit when administered to an individual. While the grape-derived component, itself, is considered an active agent due to, for example, its anti-oxidative health benefits, a variety of other active agents may also be included in the effervescent composition. The active agent preferably provides added health benefits and, most preferably, synergism. As it is often desirable to supplement ones daily diet with more than one vitamin or mineral, incorporation of those additional components into the effervescent compositions decreases the need to administer multiple different medicaments and supplements.

Useful active agents include vitamins, amino acids, pharmaceutical agents, minerals, dietary supplements, and combinations thereof. The active agent is oil-soluble. The active agent is water-soluble, substantially water-dispersible, or a combination of the two. Preferably, the active agent is selected from vitamin C and vitamin E.

Suitable vitamins include, e.g., ascorbic acid (vitamin C), biotin, carnitine, choline, folic acid, lipoic acid, nicotinic acid, pantothenic acid, pyridoxine, riboflavin, thiamin, vitamin A, vitamin B₁₂, vitamin D, vitamin E, vitamin K, and coenzymes thereof, and alpha-, beta-, and gamma-carotenes. Examples of coenzymes include biocytin, 11-cis-retinal, coenzyme B₁₂, 1,25-dihydroxycholecalciferol, flavin adenine dinucleotide, flavin mononucleotide, lipoyllysine, nicotinamide adenine dinucleotide, nicotinamide adenine dinucleotide phosphate coenzyme A pyridoxal phosphate, tetrahydrofolic acid, thiamine pyrophosphates, and mixtures thereof.

Suitable amino acids include, e.g., alanine, arginine, asparagine, cysteine, histidine, isoleucine, glutamine, glycine, leucine, lysine, methionine, ornithine, phenylalanine, praline, serine, taurine, threonine, tryptophan, L-tyrosine, valine, and mixtures thereof.

Suitable pharmaceutical agents include, e.g., antacids, analgesics, anorexics, antiasthmatics, antibiotics, antidiuretics, antiflatuents, antihistamines, antihyperactives, antihypertensives, anti-inflammatories, antimicrobials, antimigraine agents, antipyretics, antispasmodics, beta blockers, decongestants, laxatives, peptides, proteins, oligonucleotides, sedatives, tranquilizers, and mixtures thereof.

Examples of minerals include calcium, chromium, copper, iodine, iron, magnesium, phosphorus, selenium, zinc, and mixtures thereof.

Suitable dietary supplements include, e.g., acetyl-L-camitine, alpha-glycerylphosphorylcholine, amino-acids, bee pollen, bran, chondroitin, docosahexaenoic acid, fish oils (e.g., cod liver oil), ginseng, glucosamine, kelp, methylsulfonylmethane, minerals, proteins, vitamins, wheat germ, and salts and/or mixtures thereof.

Surprisingly, effervescent compositions of the invention are obtainable even when oil-soluble active agents (e.g., oil-soluble vitamins such as vitamin A, vitamin D, vitamin E, and vitamin K) are utilized. Despite the inherent difficulty associated with formulating oil-soluble components into effervescent compositions, it has been found that suitable effervescent compositions are obtainable when formulating effervescent compositions according to the invention.

Binder

The composition can also include a binder. When producing tablets, a binder assists in obtaining and maintaining the tablet form. Any suitable component can be used as a binder, provided the component promotes cohesion of the composition prior to its effervescent dissolution when contacted with a liquid. Examples of suitable binders include starches (including pregelatinized starches), natural gums, cellulose gums, microcrystalline cellulose, methylcellulose, cellulose ethers, sodium carboxymethylcellulose, ethylcellulose, gelatin, pectins, dextrose, lactose, sucrose, sorbitol, mannitol, polyethylene glycol, polyvinylpyrrolidone, alginates, polyacrylamides, polyvinyloxoazolidone, polyvinylalcohols, and mixtures thereof.

When the composition is to be tableted, the composition preferably includes a sufficient amount of binder to assist in holding the components of the composition together in the form of a tablet. Preferably binder is present in the composition in an amount of from about 10% by weight to about 60% by weight, from about 15% by weight to about 50% by weight, or even from about 25% by weight to about 40% by weight.

Lubricant

The composition can also include lubricant. When tableting the effervescent composition, a lubricant assists in the molding of the tablets. The composition can include any suitable lubricant. Although many lubricants are known, including, e.g., water-dispersible, water-soluble, water-insoluble lubricants, and combinations thereof, preferred lubricants are water-soluble. Useful water-soluble lubricants include, e.g., sodium benzoate, polyethylene glycol, L-leucine, adipic acid, and combinations thereof.

The composition preferably includes a sufficient amount of lubricant to enable the composition to be formed into tablets and released from a high speed tableting press in the form of a tablet. The effervescent composition preferably includes water-soluble lubricant in an amount of from about 1% by weight to about 15% by weight, from about 1% by weight to about 12% by weight, from about 2% by weight to about 10% by weight, or even from preferably about 3% by weight to about 8% by weight.

A useful effervescent tablet composition includes at least two water-soluble lubricants. Preferably the effervescent composition includes sodium benzoate in an amount of from about 1% by weight to about 3% by weight, and polyethylene glycol in an amount of from about 1% by weight to about 5.5% by weight.

Although not preferred, in addition to the inclusion of at least one water-soluble lubricant, the composition can also include water-insoluble lubricants including, e.g., stearates (e.g., magnesium stearate, calcium stearate and zinc stearate), oils (e.g., mineral oil, hydrogenated and partially hydrogenated vegetable oils, and cotton seed oil) and combinations thereof. Other water-insoluble lubricants include, e.g., animal fats, polyoxyethylene monostearate, talc, magnesium oxide and combinations thereof.

When present, the composition preferably includes less than about 3% by weight water-insoluble lubricants, less than about 0.6% by weight water-insoluble lubricants, or even no greater than about 0.1% by weight water-insoluble lubricants. The composition also preferably includes no greater than about 1% by weight, no greater than about 0.5% by weight, or even no greater than about 0.3% by weight oil. Most preferably the composition is free of oil and other water-insoluble lubricants.

Other Components

The composition can also include other ingredients including, e.g., coloring agents such as dyes and pigments, flavoring agents, fillers, sweeteners, surfactants (e.g., polysorbate 80 and sodium lauryl sulfate), and combinations thereof. Those of skill in the art will readily recognize that other components may be desired or utilized without detracting from the overall benefits provided by the grape-derived components and optional active agents in effervescent compositions of the invention.

Useful flavoring agents include natural and synthetic flavoring sources including, e.g., volatile oils, synthetic flavoring oils, flavoring aromatics, oils, liquids, oleoresins and extracts derived from plants, leaves, flowers, fruits, stems and combinations thereof. Useful flavoring agents include, e.g., citric oils (e.g., lemon, orange, grape, lime and grapefruit) and fruit essences (e.g., apple, pear, peach, grape, strawberry, raspberry, cherry, plum, pineapple, apricot, and other fruits). Other useful flavoring agents and their respective flavors include, e.g., aldehydes and esters (e.g., benzaldehyde (cherry, almond)), citral (e.g., alpha-citral (lemon, lime)), neral (e.g., beta-citral (lemon, lime)), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin) and mixtures thereof.

Useful coloring agents include, e.g., food, drug and cosmetic (FD&C) colorants such as dyes, lakes, and certain natural and derived colorants. Useful lakes include, for example, dyes absorbed on aluminum hydroxide and other suitable carriers.

Sweeteners can also be added to the effervescent composition. Examples of such sweeteners include cyclamic acid and its various salts, dihydrochalcone, dipeptide sweeteners (e.g., aspartame), glycyrrhizin, saccharin and its various salts (e.g., sodium and calcium salt of saccharin), and sugar alcohols (e.g., mannitol, sorbitol, sorbitol syrup, and xylitol), sugars (e.g., fructose, glucose, invert sugar, sucrose, and mixtures thereof), and combinations thereof.

Use

The effervescent composition can be provided in a wide variety of forms including, e.g., tablet, capsule, pellet, pill, composite, and free-flowing granular (e.g., powder) form. The effervescent composition preferably is provided as a single dose in an individual package.

Preferably, the dosage is formulated such that when administered on a daily basis the grape-derived component present therein provides a polyphenol content that is equivalent to the amount of polyphenol present in from about 0.5 liters (L) to about 1.0 L of red wine. Preferably each dosage form includes at least about 100 mg, at least about 200 mg, or even at least about 300 mg polyphenol.

The sum of the weight of the grape-derived component and the weight of the optional active agent present in the effervescent composition preferably is at least about 10% by weight, at least about 15% by weight, or even at least about 20% by weight. Within these preferred parameters, it is preferred that weight of the grape-derived component is greater than weight of the active agent. Preferably the grape-derived component provides at least about 55% by weight, at least about 70% by weight, or even at least about 85% by weight of the overall active agent present in the composition.

The effervescent composition can be packaged in any suitable packaging and preferably is packaged in a moisture proof, airtight, sealed metal foil pouch. One convenient dosage that is known in the industry as a “sachet” includes the effervescent composition in the form of a powder disposed in an airtight sealed package.

The effervescent composition can also be stored as a tablet in a moisture-proof package. Effervescent tablets for use in preparing beverages for individual consumption typically weigh from about 200 mg to about 5,000 mg, and can have a variety of different weights including, e.g., about 300 mg, about 500 mg, about 1,000 mg, about 2,500 mg, and about 5,000 milligrams. The tablet preferably exhibits a hardness of at least about 3 kilopounds (kP). The effervescent composition can be tabletted to a variety of degrees of hardness including, e.g., at least about 6 kP, from about 6 kP to about 10 kP, or even from about 6 kP to about 8 kP, as measured on a standard hardness tester (e.g., tablet hardness testers available from Dr. Schleuniger Pharmatron, Inc., Germany). The tablets preferably disintegrate when placed in water having a temperature of about 25° C. in less than five minutes, less than four minutes, less than 3 minutes, or even less than 2.5 minutes. The effervescent composition preferably disintegrates to form a dispersion, preferably a solution, e.g., an opaque red wine colored solution.

The effervescent composition in tablet form can be administered by placing the effervescent composition in an aqueous liquid to form a beverage. The effervescent composition disperses readily in an aqueous liquid (e.g., water), and preferably dissolves in water to form a clear solution that is essentially free of particulate scum. The effervescent composition can be added to any suitable aqueous liquid including, e.g., water, alcohol, fruit juice, milk, other aqueous liquids and combinations thereof. Preferably, the liquid is water. The amount of liquid needed to adequately dissolve the effervescent composition depends on the amount of effervescent composition and the taste and consistency desired by the consumer. Preferably, the individual components of the effervescent composition are formulated to provide a palatable composition in a convenient dosage form.

The invention will now be described by way of the following non-limiting examples.

### EXAMPLES

### Example 1

An effervescent composition was prepared by combining the ingredients in the proportional amounts (reported in milligrams (mg) unless reported otherwise) set forth in Table 1 with mixing. Upon formulation, the composition was in granular form having a grayish/burgundy color. The formulation was mixed using a mechanical mixer for 10 minutes, after which time the formulation was transferred to a tablet press having a one-inch tool to form tablets each weighing from approximately 4,950 mg to 5,050 mg. The tablets were each pressed to a hardness of approximately six to seven kilopounds and a thickness of about 0.27 in to 0.28 in. The tablets formed had an eye-pleasing burgundy speckled pattern.

The tablets were then placed in excess water, approximately 200 mL, and the amount of time required to achieve 100% disintegration was measured. The presence or absence of particulate scum on the surface of the solution so formed and the clarity of the solution were observed and noted. The results are reported in Table 1.

TABLE 1

| Compound | 1 |
|---|---|
| Sodium bicarbonate | 1,200.0 |
| (powder) | |
| Citric acid (fine) | 1,300.0 |
| Sodium benzoate (325 | 100.0 |
| mesh) | |
| PEG* 3350 | 200.0 |
| Stevia | 40.0 |
| Sorbitol/ | 700 |
| Mannitol blend | |
| Provinols Red Wine | 100.0 |
| Extract | |
| Ascorbic acid | 1,000.0 |
| Vitamin A (250 CWS) | 22.0 |
| Vitamin E (50% SD) | 66.0 |
| Vitamin B1 (Thiamine | 1.5 |
| Mononitrate) | |
| Vitamin B2 (Riboflavin- | 2.3 |
| 5-phosphate) | |
| Vitamin B3 | 20.0 |
| (Niacinamide) | |
| Vitamin B5 (Calcium | 10.0 |
| Pantothenate) | |
| Vitamin B6 (Pyridoxine | 2.0 |
| HCl) | |
| Vitamin B12 | 0.6 |
| (Cyanocobalamin 1%) | |
| Magnesium Sulfate | 200.0 |
| Zinc Sulfate | 37.0 |
| Average Tablet Weight | 5.0 |
| (mg) | |
| Average Tablet Hardness | 5.6 |
| (Kp) | |
| Dissolution Time | 2 minutes, 15 seconds |
| Visual Observations | Dark burgundy colored liquid |
| | that is slightly hazy; lots of |
| | particulate around surface |
| | edges; slight wine-like odor; |
| | sweet pleasant taste |

*PEG = polyethylene glycol

### Examples 2-3

Effervescent compositions were prepared by combining the ingredients in the proportional amounts (reported in milligrams (mg) unless reported otherwise) set forth in Table 2 with mixing. Each formulation was mixed using a V-blender for 10 minutes (220 revolutions), after which time the formulation was transferred to a tablet press to form tablets. Tableting of both formulations of 2 and 3 was found to work particularly well.

TABLE 2

| Compound | 2 | 3 |
|---|---|---|
| Sodium | 750.0 | 750.0 |
| Bicarbonate #5 | | |
| Citric Acid | 1,000.0 | 1,000.0 |
| Sodium Benzoate (325 | 75.0 | 75.0 |
| mesh) | | |
| PEG* 3350 | 120.0 | 120.0 |
| Sodium Carbonate | 100.0 | 100.0 |
| Grade 50 | | |
| Sorbitol Instant | 650.0 | 650.0 |
| MegaNatural ™ Gold | 345.0 | 345.0 |
| Grape Seed Extract | | |
| (Polyphenolics) | | |
| Ace K | 30.0 | 30.0 |
| Artificial Cranberry | 50.0 | 50.0 |
| Flavor SD #705K | | |
| (Virginia Dare) | | |
| Natural Grapefruit | 10.0 | 10.0 |
| Flavor | | |
| Abscorbic Acid (99% | — | 60.6 |
| Pure) | | |
| Vitamin E (500 IU/g) | — | 60.6 |
| Average Tablet weight | 3.2 | 3.3 |
| (g) | | |
| Average Tablet | 5.4 | 7.6 |
| Hardness | | |
| Dissolution Time | 3 minutes 10 seconds | 3 minutes 30 seconds |
| Observations | Opaque brown | Opaque brown |
| | solution. Foamy. | solution. Foamy. |
| | Grape seed did not | Grape seed did not |
| | dissolve. Viscous | dissolve. Viscous |
| | glue-like grape | glue-like grape |
| | seed on surface. | seed on surface. |

*PEG = polyethylene glycol

### Example 4

The effervescent composition was prepared by combining the ingredients in the proportional amounts (reported in milligrams (mg) unless reported otherwise) set forth in Table 3 with mixing. The formulation was mixed using a V-blender for 10 minutes (220 revolutions), after which time the formulation was transferred to a tablet press to form tablets.

TABLE 3

| Compound | 4 |
|---|---|
| Sodium Bicarbonate #5 | 750.0 |
| Citric Acid | 900.0 |
| Sodium Benzoate (325 mesh) | 60.0 |
| PEG* 3350 | 100.0 |
| Sodium Carbonate Grade 50 | 100.0 |
| Sorbitol Instant | 800.0 |
| Red Wine Extract (Brucia Plant Extracts, a | 300.0 |
| division of Naturex, Inc. (Shingle Springs, | |
| CA)) | |
| Ace K | 10.0 |
| Artificial Cranberry Flavor SD #705K (Virginia | 10.0 |
| Dare) | |
| Natural Grapefruit Flavor | 5.0 |
| Average Tablet weight (mg) | 3.0 |
| Average Tablet Hardness (Kp) | 4.6 |
| Dissolution time | 1 minute |
| Observations | Particulate present. |
| | Extract floats on |
| | surface. Some |
| | particulates on bottom. |

*PEG = polyethylene glycol

### Examples 5-6

Effervescent compositions were prepared by combining the ingredients in the proportional amounts (reported in milligrams (mg) unless reported otherwise) set forth in Table 4 with mixing. Each formulation was mixed using a V-blender for 15 minutes (330 revolutions), after which time the formulation was transferred to a tablet press to form tablets.

TABLE 4

| Compound | 5 | 6 |
|---|---|---|
| Sodium Bicarbonate #5 | 750.0 | 750.0 |
| Citric Acid | 900.0 | 900.0 |
| Sodium Benzoate (325 mesh) | 60.0 | 60.0 |
| PEG* 3350 | 100.0 | 100.0 |
| Sodium Carbonate Grade 50 | 100.0 | 100.0 |
| Sorbitol Instant | 800.0 | 800.0 |
| MegaNatural ™ Red Wine | 500.0 | 1,000.0 |
| Concentrate, Code GW3050 | | |
| (Polyphenolics, a division of | | |
| Canandaigua Wine Company, Inc. | | |
| (Madera, CA)) | | |
| Ace K | 10.0 | 10.0 |
| Artificial Cranberry | 10.0 | 10.0 |
| Flavor SD #705K | | |
| (Virginia Dare) | | |
| Natural Grapefruit Flavor | 5.0 | 5.0 |
| Average Tablet weight (mg) | 3.1 | 3.3 |
| Average Tablet Hardness (Kp) | 6.7 | 5.9 |
| Dissolution time | 2 minutes, 2 | Other than a tiny |
| | seconds | core, the tablet |
| | | dissolved in 4 |
| | | minutes, the tiny |
| | | core dissolved |
| | | after a total |
| | | of six minutes |
| Observations | Foamy, opaque | Foamy, opaque |
| | deep red | deep red |
| | solution | solution |
| 3 Days Storage at Room Temp. | No puffing. | No puffing. |
| | Tablet moves. | Tablet moves. |
| 3 Days Storage at | Slight puffing. | Slight puffing. |
| 40° C./75% Relative | Tablet moves. | Tablet moves. |
| Humidity | | |
| 3 Days Storage at 45° C. | Slight puffing. | Slight puffing. |
| | Tablet moves. | Tablet moves. |
| 7 Days Storage at Room Temp. | No puffing. No | No puffing. |
| | Tablet | No Tablet |
| | Movement. | Movement. |
| 7 Days Storage at | Acceptable | Puffing. Tablet |
| 40° C./75% Relative | puffing. Tablet | Movement |
| Humidity | Movement. | |
| 7 Days Storage at 45° C. | Acceptable | Fail. Considerable |
| | puffing. Tablet | puffing. |
| | Movement | Considerable |
| | | tablet movement. |

*PEG = polyethylene glycol

Other embodiments are within the claims. Various modifications and alterations of the invention will become apparent to those skilled in the art without departing from the spirit and scope of the invention, which is defined by the accompanying claims. It should be noted that steps recited in any method claims below do not necessarily need to be performed in the order that they are recited. Those of ordinary skill in the art will recognize variations in performing the steps from the order in which they are recited.

What is claimed is:

1. An effervescent tablet comprising:
at least one grape-derived component comprising greater than 100 milligrams polyphenols;
at least one effervescent agent;
binder;
lubricant; and
optionally at least one active agent,
wherein at least 10% by weight of the tablet is the sum of the weight of the grape-derived component and the weight of the optional active agent.

2. The effervescent tablet of claim 1, wherein the grape-derived component comprises at least about 200 milligrams polyphenols.

3. The effervescent tablet of claim 1, wherein the grape-derived component comprises at least about 300 milligrams polyphenols.

4. The effervescent tablet of claim 1, wherein the grape-derived component is selected from grape seed extract, grape skin extract, grape juice extract, red wine extract, and combinations thereof.

5. The effervescent tablet of claim 1, wherein the grape-derived component is derived from fermented grapes.

6. The effervescent tablet of claim 1, wherein the grape-derived component comprises at least about 25% dry weight polyphenols.

7. The effervescent tablet of claim 1, wherein the grape-derived component comprises at least about 75% dry weight polyphenols.

8. The effervescent tablet of claim 1, wherein the grape-derived component comprises at least about 1.5% dry weight anthocyanins.

9. The effervescent tablet of claim 1, wherein the grape-derived component comprises at least about 70% of the sum of the weight of the grape-derived component and the weight of the active agent.

10. The effervescent tablet of claim 1, wherein the grape-derived component comprises at least about 85% of the sum of the weight of the grape-derived component and the weight of the active agent.

11. The effervescent tablet of claim 1, comprising an active agent selected from vitamins, amino acids, pharmaceutical agents, minerals, dietary supplements, and combinations thereof.

12. The effervescent tablet of claim 1, comprising an oil-soluble active agent.

13. The effervescent tablet of claim 1, wherein said active agent comprises vitamin E.

14. The effervescent tablet of claim 1, comprising an active agent selected from vitamin C and vitamin E.

15. The effervescent tablet of claim 1, wherein the tablet has a hardness of at least about 6 kilopounds and disintegrates in water having a temperature of about 25° C. in less than about 4 minutes.

16. The effervescent tablet of claim 1, wherein the grape-derived component comprises red wine extract, and the composition disintegrates in water to an opaque solution.

17. An effervescent composition comprising:
at least one grape-derived component;
at least one effervescent agent; and
optionally at least one active agent,
wherein at least 10% by weight of the composition is the sum of the weight of the grape-derived component and the weight of the active agent, and
the weight of the grape-derived component being greater than weight of the active agent.

18. The effervescent composition of claim 17, wherein the grape-derived component comprises at least about 25% dry weight polyphenols.

19. The effervescent composition of claim 17, wherein the grape-derived component comprises at least about 75% dry weight polyphenols.

20. The effervescent composition of claim 17, wherein the grape-derived component comprises red wine extract, and the composition disintegrates in water to an opaque solution.

21. A tablet comprising the effervescent composition of claim 17.

22. An airtight sealed package comprising:
a sachet; and
an effervescent powder disposed in said sachet, said effervescent powder comprising
at least two grape-derived components,
at least one effervescent agent, and
optionally at least one active agent.

23. The sachet of claim 22, wherein the sum of the grape-derived components and the active agent comprises at least about 10% by weight of the effervescent powder.

24. A method of using the effervescent composition of claim 1, the method comprising combining the effervescent composition and an aqueous liquid to form a beverage.

25. A method of using the effervescent composition of claim 17, the method comprising combining the effervescent composition and an aqueous liquid to form a beverage.
